(19) United States
(12) Patent Application Publication
(10) Pub. No.: US 2025/0002437 A1
(43) Pub. Date: Jan. 2, 2025

(54) REMOVAL OF ALDEHYDES IN ACETIC ACID PRODUCTION

(71) Applicant: LyondellBasell Acetyls, LLC, Houston, TX (US)

(72) Inventor: Noel C. Hallinan, Loveland, OH (US)

(73) Assignee: LyondellBasell Acetyls, LLC, Houston, TX (US)

(21) Appl. No.: 18/754,473

(22) Filed: Jun. 26, 2024

Related U.S. Application Data
(60) Provisional application No. 63/511,353, filed on Jun. 30, 2023.

(51) Int. Cl. C07C 51/12; B01J 20/12; B01J 20/28; C07C 51/44

(52) U.S. Cl. CPC C07C 51/12; B01J 20/12; B01J 20/28059; B01J 20/28061; B01J 20/28064; B01J 20/28071; B01J 20/28073; B01J 20/28076; C07C 51/44

(57) ABSTRACT

Processes for producing carboxylic acid are included herein. The processes include contacting methanol and carbon monoxide in the presence of a liquid reaction medium under carbonylation conditions sufficient to form a carbonylation product comprising acetic acid and acetaldehyde. At least a portion of the carbonylation product or a derivative thereof is contacted with a phyllosilicate clay-based adsorbent at adsorption conditions sufficient to selectively reduce a concentration of acetaldehyde present in the carbonylation product or a derivative thereof.

### CROSS-REFERENCE TO RELATED APPLICATIONS

The application claims the benefit of priority to U.S. Provisional Patent Application No. 63/511,353 filed on Jun. 30, 2023, which is incorporated herein by reference in its entirety.

### FIELD OF THE INVENTION

This disclosure relates to the production of acetic acid. More particularly, the disclosure relates to removal of acetaldehyde in acetic acid production.

### BACKGROUND OF THE INVENTION

In the current acetic acid production process, a reaction mixture is withdrawn from a reactor and is separated in a flash tank into a liquid fraction and a vapor fraction comprising acetic acid generated during the carbonylation reaction. The liquid fraction may be recycled to the carbonylation reactor, and the vapor fraction is passed to a separations unit, which by way of example may be a light-ends distillation column. The light-ends distillation column separates a crude acetic acid product from other components. The crude acetic acid product is passed to a drying column to remove water and then is subjected to further separations to recover acetic acid.

One challenge facing the industry is the presence of aldehyde(s) in acetic acid production, which can be present in the feed and is also formed as an undesired byproduct of carbonylation reactions. Processes for removing aldehydes exist; however, there continues to be a need to improve upon, and provide alternatives to, current aldehyde removal processes.

### SUMMARY OF THE INVENTION

The present disclosure relates to the production of acetic acid and related processes. In some embodiments, a process for the production of acetic acid comprises reacting methanol and carbon monoxide in the presence of a carbonylation catalyst to form acetic acid in an acetic acid production reactor. The process further comprises flashing a reaction mixture discharged from the acetic acid production reactor into a vapor stream and a liquid stream, wherein the vapor stream comprises acetic acid, water, methanol, methyl acetate, methyl iodide, and acetaldehyde. The vapor stream is separated by distillation in a distillation column into: a product side stream comprising acetic acid and water; a bottoms stream; and an overhead stream comprising methyl iodide, water, methyl acetate, acetic acid, and acetaldehyde. The overhead stream in condensed into: a light aqueous phase stream, comprising methyl iodide, acetaldehyde, water, methyl acetate, and acetic acid; and a heavy organic phase stream, comprising methyl iodide, acetaldehyde, water, methyl acetate, and acetic acid. An intermediate process stream is contacted with a phyllosilicate clay-based adsorbent at adsorption conditions sufficient to produce a treated intermediate process stream, wherein the intermediate process stream comprises at least a portion of the light aqueous phase stream, at least a portion of the heavy organic phase stream, or a combination thereof. The intermediate process stream has a first acetaldehyde content, the treated intermediate process stream has a second acetaldehyde content, and the second acetaldehyde content is less than the first acetaldehyde content. In some embodiments, the process further comprises recycling the treated intermediate process stream to the acetic acid production reactor.

In some embodiments, a method for removing acetaldehyde from an acetic acid system, comprises providing from the acetic acid system a solution, comprising acetic acid, water, methyl acetate, methyl iodide, and acetaldehyde, wherein the acetaldehyde is present in a first concentration based on the total weight of the solution. The solution is contacted with a phyllosilicate clay-based adsorbent at adsorption conditions sufficient to produce a treated solution, wherein the solution has a first acetaldehyde content, the treated solution has a second acetaldehyde content, and the second acetaldehyde content is less than the first acetaldehyde content.

The above paragraphs present a simplified summary of the presently disclosed subject matter in order to provide a basic understanding of some aspects thereof. The summary is not an exhaustive overview, nor is it intended to identify key or critical elements to delineate the scope of the subject matter claimed below. Its sole purpose is to present some concepts in a simplified form as a prelude to the more detailed description set forth below.

### BRIEF DESCRIPTION OF THE DRAWINGS

The claimed subject matter may be understood by reference to the following description taken in conjunction with the accompanying drawings, in which like reference numerals identify like elements, and in which:

FIG. 1 is a schematic of an exemplary acetic acid production system according to an embodiment of the invention;

FIG. 2 shows overlaid graphs of FTIR absorbance for montmorillonite slurried with an HAc/octane solution for different reaction times according to an embodiment of the invention;

FIG. 3 shows overlaid graphs of FTIR absorbance for dried montmorillonite slurried with an HAc/octane solution for different reaction times according to an embodiment of the invention;

FIG. 4 shows overlaid graphs of FTIR absorbance peak height for acetaldehyde and paraldehyde (PA) vs. time for montmorillonite and dried montmorillonite with an HAc/decane solution according to an embodiment of the invention;

FIG. 5 shows overlaid graphs of FTIR absorbance for a solid acid catalyst slurried with an HAc/octane solution for different reaction times;

FIG. 6 shows overlaid graphs of FTIR absorbance for various clays slurried with a crotonaldehyde (CA)/octane solution; and

FIG. 7 shows overlaid graphs of FTIR absorbance HAc/octane solution and neat water for different reaction times.

While the disclosed process and system are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

A detailed description of embodiments of the disclosed process follows. However, it is to be understood that the described embodiments are merely exemplary of the process and that the process may be embodied in various and alternative forms of the described embodiments. Therefore, specific procedural, structural and functional details which are addressed in the embodiments described herein are not to be interpreted as limiting, but merely as a basis for the claims and as a representative basis for teaching one skilled in the art to variously employ the disclosed process.

The embodiments illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations disclosed herein is to be understood to set forth every number and range encompassed within the broader range of values. It is to be noted that the terms “range” and “ranging” as used herein generally refer to a value within a specified range and encompasses all values within that entire specified range.

The designation of groups of the Periodic Table of the Elements as used herein is in accordance with the current IUPAC convention. The expression “HAc” is used herein as an abbreviation for acetaldehyde. The expression “MeI” is used herein as an abbreviation for methyl iodide. The expression “HI” is used herein as an abbreviation for hydrogen iodide. The expression “acac” is used herein as an abbreviation for acetoacetate anion, i.e., H₃CC(═O)CH₂C(═O)O—. Unless specifically indicated otherwise, the expression “wt %” as used herein refers to the percentage by weight of a particular component in the referenced composition. With respect to all ranges disclosed herein, such ranges are intended to include any combination of the mentioned upper and lower limits even if the particular combination is not specifically listed.

Embodiments of the disclosed process and system involve the production of acetic acid by carbonylating methanol in a carbonylation reaction. The carbonylation reaction may be represented by: CH₃OH+CO→CH₃COOH

### Acetic Acid Production

Embodiments described herein include processes for producing acetic acid. Furthermore, one or more specific embodiments include production of glacial acetic acid (which is encompassed by the term “acetic acid” as referenced herein). Glacial acetic acid refers to acetic acid that is often undiluted (includes a water concentration of up to about 0.15 wt % based on the total weight of acetic acid and water). In one or more embodiments, the acetic acid production processes may include carbonylation processes. For example (and for purposes of discussion herein), the acetic acid production processes may include the carbonylation of methanol and/or its derivatives to produce acetic acid.

The carbonylation processes utilized to produce acetic acid often include reacting an alcohol, such as methanol, with carbon monoxide in the presence of a reaction medium, such as a liquid reaction medium, under carbonylation conditions sufficient to form a carbonylation product, including acetic acid and recovering the formed acetic acid from the carbonylation product. As described herein, the term “liquid reaction medium” refers to a reaction medium that is primarily liquid in form. For example, the liquid reaction medium contains minor amounts of alternative phases. In one or more embodiments, the liquid reaction medium is at least 90%, or at least 91%, or at least 92%, or at least 93%, or at least 94%, or at least 95%, or at least 96%, or at least 97%, or at least 98%, or at least 99% liquid phase.

In some embodiments, the carbonylation product includes the formed acetic acid. In addition to the acetic acid, the carbonylation product often includes one or more impurities. Impurities are defined herein as any component in a process stream other than the targeted product itself (e.g., acetic acid is the targeted product in the carbonylation product stream). For example, the impurities present in carbonylation product stream may include water, aldehydes (e.g., acetaldehyde, crotonaldehyde, butyraldehyde and derivatives thereof), alkanes, formic acid, methyl formate or combinations thereof as well as additional compounds other than the acetic acid, depending on the specific process.

The separation of impurities from the acetic acid prior to use of the acetic acid in subsequent processes such as industrial processes is often preferred or necessary. Such separation processes may include those available in the relevant literature and may include separating one or more of the impurities from the acetic acid within a process stream (wherein the process stream may be referred to as “impure acetic acid”) to form purified acetic acid via one or more methods, including, but not limited to, extraction, distillation, extractive distillation, caustic treatment, scavenging, adsorption, and combinations thereof. As used herein, the term “purified acetic acid” refers to an acetic acid stream having a concentration of one or more impurities that is reduced in comparison to that impurity's concentration in the impure acetic acid. It is to be noted that use of the term “acetic acid stream” herein refers to any stream containing acetic acid.

While many processes exist for the separation of the impurities, such processes can be difficult to implement, are less effective than desired, and/or are costly. Thus, continuous efforts have been underway to improve and develop methods to separate these impurities from acetic acid. Embodiments described herein provide separation of one or more impurities via selective adsorption.

Thus, one or more embodiments include contacting at least a portion of the carbonylation product (or a derivative thereof) with a phyllosilicate clay-based adsorbent at adsorption conditions sufficient to selectively reduce the concentration of one or more impurities present in the carbonylation product. As used herein, the term “selectively reduce” refers to the reduction in concentration of one or more target impurities (e.g., aldehydes, or more specifically acetaldehyde) without substantial reduction in the concentration of acetic acid present in the stream. As used herein, the term “substantial” references a value that does not change by more than 0.5%.

In one or more embodiments, the impure acetic acid stream may include acetaldehyde at a concentration in a range of 0 wt % to 4.5 wt %, or at least 3 wt %, or 2.5 wt % to 3.5 wt % based on the total weight of the impure acetic acid stream. The impure acetic acid stream may include formic acid at a concentration in a range of from 0 wt % to 4.5 wt %, or at least 3 wt %, or 2.5 wt % to 3.5 wt % based on the total weight of the impure acetic acid stream. In one or more specific, non-limiting embodiments, the impure acetic acid stream may include water at a concentration in a range of 0 wt % to 2.5 wt %, or at least 1.25 wt %, or 1.25 wt % to 2 wt % based on the total weight of the impure acetic acid stream.

At least a portion of the carbonylation product contacts the phyllosilicate clay-based adsorbent under adsorption conditions sufficient to selectively reduce the concentration of one or more components present in the carbonylation product. For example, the concentration of one or more of the impurities, such as acetaldehyde, may be reduced by at least 50%, or at least 80%, or at least 85%, or at least 90%, or at least 95%, or at least 98%. In some embodiments, the purified acetic acid stream may include acetaldehyde at a concentration of less than 2 wt %, or less than 1.5 wt %, or less than 1 wt % based on the total weight of the purified acetic acid stream. In certain embodiments, the purified acetic acid stream may include formic acid at a concentration of less than 2 wt %, or less than 1.5 wt %, or less than 1 wt % based on the total weight of the purified acetic acid stream. In further embodiments, the purified acetic acid stream may include water at a concentration of less than 1 wt %, or less than 0.75 wt %, or less than 0.5 wt % based on the total weight of the purified acetic acid stream.

In some embodiments, the phyllosilicate clay-based adsorbent comprises a kaolinite, a smectite, a vermiculite, an illite, a chlorite, or a combination thereof. In some embodiments, the phyllosilicate clay-based adsorbent comprises a smectite. In some embodiments, the smectite comprises a montmorillonite, beidellite, nantronite, saponite, hectorite, a bentonite, or a combination thereof. In some embodiments, the smectite comprises a montmorillonite, a bentonite, or a combination thereof. In some embodiments, the phyllosilicate clay-based adsorbent comprises a natural clay, a synthetic clay, or a combination thereof.

In some embodiments, the phyllosilicate clay-based adsorbent comprises interlayer cations, interlayer metal-oxide pillars, or a combination thereof.

In some embodiments, the phyllosilicate clay-based adsorbent comprises a layered structure in which tetrahedral and octahedral sheets of silica and/or alumina are separated by an interlayer space occupied by metal cations such as Na+ and by H₂O. This interlayer spacing can be varied by a technique called pillaring in which a poly oxo metal cation is introduced and acts as a pillar between layers, forcing them apart, and thus forming a pillared clay. By varying the metal in the pillaring agent, a wide range of interlayer spacings can be achieved.

In some embodiments, the phyllosilicate clay-based adsorbent comprises pillared aluminosilicate quaternary ammonium glycolate smectite. Pillared AQGS clays are produced by treating natural clays with a solution of quaternary ammonium glycolate salts, which can intercalate between the clay layers and expand the interlayer spacing. The expanded interlayer spacing creates a porous structure that can trap and adsorb molecules from a solution. In some embodiments, the properties of pillared AQGS clays are improved by utilizing “pillars” comprising metal ions such as aluminum, titanium, or zirconium. In such embodiments, the pillaring process involves adding a solution of the metal ions to the interlayer space of the clay, which creates a more stable structure with increased surface area and porosity. The resulting pillared AQGS clays have a high surface area, high porosity, and a uniform pore size distribution, which make them effective absorbents.

In some embodiments, the phyllosilicate clay-based adsorbent is pretreated with acid or alkali mixtures to exchange some or all of the ions normally present in the untreated clay. In some embodiments, these pillared clays can be acidified to modify their performance as an adsorbent. Clay-based adsorbents can be acidified through a process called acid activation. Acid activation involves treating the clay with an acid solution, typically a strong mineral acid such as hydrochloric acid (HCl) or sulfuric acid (H₂SO₄). This treatment modifies the clay's properties, creating a more porous and reactive surface, which enhances its adsorption capacity.

In some embodiments, acid treatment is performed on a clay that has a layered structure and high cation exchange capacity (CEC) to ensure good adsorption properties, such as, but not limited to, a bentonite, a montmorillonite, or a combination thereof. The clay can be mixed with a strong mineral acid solution, such as hydrochloric acid or sulfuric acid. The acid concentration and treatment conditions may vary depending on the specific clay and desired properties. In some embodiments, the clay is mixed with the acid solution at a certain ratio (e.g., 1:1) and stirred or agitated for a time period sufficient to achieve the desired level of activation. The acid reacts with the clay, causing an exchange of cations and the dissolution of some components, in turn leading to the creation of additional pores and an increase in surface area. After the desired reaction time, the mixture can be filtered and/or centrifuged to separate the acid solution from the clay residue. The acid-treated clay residue is thoroughly washed with water to remove any remaining acid and soluble impurities. The washing process is typically repeated several times until the pH of the wash water reaches a neutral level. After washing, the clay is usually dried under controlled conditions, such as low temperature or ambient air drying. The acid activation process modifies the structure and surface properties of the clay, resulting in an adsorbent material with enhanced adsorption capacity. The increased porosity and surface area provide more sites for adsorption, allowing the clay-based adsorbent to effectively capture and retain target impurities. The specific acid activation process may vary depending on the type of clay and the intended application of the adsorbent. Protocols for performing acid activation on clay-based adsorbents available in the open literature can be utilized and/or modified to achieve desired adsorption properties for specific process conditions.

In some embodiments, the phyllosilicate clay-based adsorbent is characterized by a porosity, a surface area, a pore size distribution, a chemical composition, a cation exchange capacity (CEC), and/or a thermal stability.

The porosity of a clay can affect its ability to absorb or adsorb different molecules. Clays with high porosity, such as vermiculites and montmorillonites, may be more effective at absorbing larger molecules, while clays with lower porosity, such as kaolinites and halloysites, may be better at absorbing smaller molecules. In some embodiments, the phyllosilicate clay-based adsorbent has a porosity as measured by pore volume in the range of from 0.1 to 1.4 cm³/g, from 0.2 to 1.2 cm³/g, or from 0.3 to 1.0 cm³/g. In some embodiments, acid activation is used to increase the porosity of the phyllosilicate clay-based adsorbent.

The surface area of a clay can also affect its ability to absorb or adsorb molecules. Clays with a higher surface area, such as montmorillonites and smectites, may be more effective at adsorbing molecules due to the greater number of active sites on the surface of the clay particles. In some embodiments, the phyllosilicate clay-based adsorbent has a surface area in the range of from 50 m²/g to 800 m²/g, from 100 m²/g to 750 m²/g, from 150 m²/g to 700 m²/g, or from 200 m²/g to 600 m²/g.

The pore size distribution of the clay can also be important for absorbent applications. Clays with a narrow pore size distribution, such as kaolinites and hectorites, may be more effective at selectively absorbing certain molecules.

The chemical composition of a clay can also be an important factor for absorbent applications. Clays with a high content of silicates, such as montmorillonites and smectites, may be more effective at adsorbing polar molecules, while clays with a high content of alumina, such as kaolinites and halloysites, may be more effective at adsorbing nonpolar molecules.

The cation exchange capacity (CEC) of a clay can affect its ability to absorb or adsorb charged molecules. Clays with a high CEC, such as montmorillonites and smectites, may be more effective at absorbing or adsorbing charged molecules due to their ability to exchange cations with the surrounding solution.

The thermal stability of the clay can also be important for absorbent applications, particularly in high-temperature environments. Clays with higher thermal stability, such as kaolinites and halloysites, may be more suitable for use as an absorbent in high-temperature environments.

In some embodiments, the phyllosilicate clay-based adsorbent has an average removal capability of greater than or equal to 0.3 g acetaldehyde/g of adsorbent, greater than or equal to 0.4 g acetaldehyde/g of adsorbent, greater than or equal to 0.5 g acetaldehyde/g of adsorbent, or greater than or equal to 0.6 g acetaldehyde/g of adsorbent.

In some embodiments, the concentration of acetaldehyde in the intermediate product stream is selectively reduced by an amount greater than or equal to 50%, greater than or equal to 60%, greater than or equal to 70%, greater than or equal to 80%, greater than or equal to 85%, greater than or equal to 90%, greater than or equal to 95%, or greater than or equal to 98%.

At least a portion of the carbonylation product may contact the phyllosilicate clay-based adsorbent via methods available in the relevant literature. For example, the phyllosilicate clay-based adsorbent may be disposed on a fixed bed and at least a portion of the carbonylation product may pass through the bed to selectively reduce the concentration of one or more components therein. The phyllosilicate clay-based adsorbent may be disposed on the fixed bed by manners available in the relevant literature. The phyllosilicate clay-based adsorbent may be loaded in the bed in an amount in a range of from 1 g to 10 g phyllosilicate clay-based adsorbent/g organic material to be adsorbed, from 2 g to 8 g phyllosilicate clay-based adsorbent/g organic material to be adsorbed, or from 4 g to 6 g phyllosilicate clay-based adsorbent/g organic material to be adsorbed. In some embodiments, the phyllosilicate clay-based adsorbent organic material selectively adsorbs acetaldehyde.

The adsorption conditions vary depending upon numerous factors. However, the phyllosilicate clay-based adsorbent conditions are such that they promote adsorption but are not sufficient to promote catalysis/reaction. Thus, in one or more embodiments, the adsorption temperature is in a range of from room temperature to 250° C., from room temperature to 225° C., or from room temperature to 200° C. As used herein, “room temperature” means that a temperature difference of a few degrees does not matter to the phenomenon under investigation. In some environments, room temperature may include a temperature in a range of about 20° C. to about 28° C., while in other environments, room temperature may include a temperature in a range of about 10° C. to about 32° C., for example. However, room temperature measurements often do not include close monitoring of the temperature of the process and therefore such a recitation does not intend to bind the embodiments described herein to any predetermined temperature range.

It is contemplated that the phyllosilicate clay-based adsorbent may occasionally require regeneration or replacement. The regeneration procedure often includes processing the spent adsorbent at room temperature or at high temperatures and may include any regeneration procedure available in the relevant literature. The phyllosilicate clay-based adsorbent may be regenerated either in the phyllosilicate clay-based adsorbent bed or may be removed from the phyllosilicate clay-based adsorbent bed for regeneration. Such regeneration is known to the skilled artisan. However, a non-limiting illustrative embodiment of in-line regeneration is described below.

In a non-limiting example of in-line regeneration, the phyllosilicate clay-based adsorbent bed is initially taken off-line and the phyllosilicate clay-based adsorbent disposed therein is purged. Off-stream reactor purging may be performed by contacting the phyllosilicate clay-based adsorbent in the off-line phyllosilicate clay-based adsorbent bed with a purging stream, which may include an inert gas, e.g., nitrogen. The off-stream reactor purging conditions are typically determined by individual process parameters and are generally known to one skilled in the art.

The phyllosilicate clay-based adsorbent may then optionally undergo a regeneration step. The regeneration conditions may be any conditions that are effective for at least partially reactivating the phyllosilicate clay-based adsorbent and are generally known to one skilled in the art. For example, regeneration may include heating the phyllosilicate clay-based adsorbent to a temperature or a series of temperatures, such as a regeneration temperature in a range of 50° C. to 200° C. above the adsorption temperature.

In order to minimize disruption to the process during periods of regeneration or replacement, one or more embodiments of the present disclosure utilize swing beds for the adsorption of one or more acetic acid processing impurities. In such embodiments, continuous operation can be achieved. For example, one phyllosilicate clay-based adsorbent bed may be taken off-line for potential removal and/or regeneration of the phyllosilicate clay-based adsorbent, while the remaining phyllosilicate clay-based adsorbent bed may remain on-line for production.

In one or more embodiments, components within the carbonylation product stream (or at least a portion thereof) may be separated from one another via flash separation into a liquid fraction and a vapor fraction. The liquid fraction may include residual carbonylation catalyst as well as other components, while the vapor fraction may include acetic acid, unreacted reactants, water, methyl iodide and impurities generated during the carbonylation reaction. The liquid fraction may be recycled to the carbonylation reaction while the vapor fraction may undergo supplemental separation.

The supplemental separation may include a first column (e.g., a light ends distillation column) adapted to separate components of the liquid fraction and form a first overhead stream and an acetic acid stream. The first overhead stream may include methyl iodide, water, methanol, methyl acetate, impurities or combinations thereof, for example. The acetic acid stream may be passed to a drying column to remove any water contained therein and then a second column (e.g., a heavy ends distillation column) adapted to separate components of the acetic acid stream and form a second overhead stream and a bottoms stream. The second overhead stream may include methyl iodide, methyl acetate, acetic acid, water, impurities or combinations thereof.

The first overhead stream may be condensed and separated in a decanter to form, relative to each phase, a “light” aqueous phase and a “heavy” organic phase. The heavy organic phase may include methyl iodide and aldehyde impurities. The light aqueous phase may include water, acetic acid, methyl acetate, and aldehyde impurities. The light aqueous phase may be recycled to the reactor or for light ends distillation. In some embodiments, the light aqueous phase or a portion thereof, the heavy organic phase or a portion thereof, or a combination thereof, containing aldehyde impurities (e.g., acetaldehyde) is contacted with the phyllosilicate clay-based adsorbent under adsorption conditions to selectively reduce such impurities.

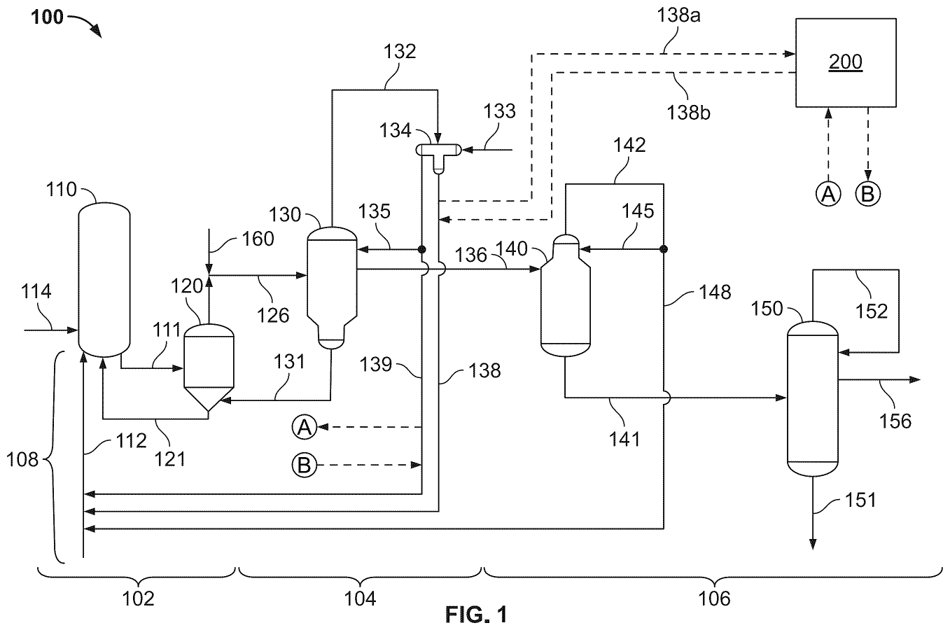

FIG. 1 is a schematic of an exemplary acetic acid production system 100 implementing the carbonylation reaction. In certain embodiments, the acetic acid system 100 may include a reaction area 102, a light-ends area 104, a purification area 106 and a recycle area 108, which correlate to specific processing equipment. Note that the “streams” discussed herein may be part of more than one functional area.

The reaction area 102 may include a reactor 110, a flash vessel 120, and associated equipment, and streams associated with the reactor 110 and the flash vessel 120. For example, the reaction area 102 may include the reactor 110, the flash vessel 120, and streams (or portions of streams) 111, 112, 114, 121, 126, 131, 160, 138, 139 and 148. The reactor 110 is a reactor or vessel in which methanol is carbonylated in the presence of a catalyst to form acetic acid at elevated pressure and temperature. The flash vessel 120 is a tank or vessel in which a reaction mixture obtained in the reactor is at least partially depressurized and/or cooled to form a vapor stream and a liquid stream. The liquid stream 121 may be a product or composition which has components in the liquid state under the conditions of the processing step in which the stream is formed. The vapor stream 126 may be a product or composition which has components in the gaseous state under the conditions of the processing step in which the stream is formed.

In some embodiments, the reactor 110 may be configured to receive a carbon monoxide feed stream 114 and a methanol feed stream 112. A reaction mixture may be withdrawn from the reactor in stream 111. Other streams may be included such as, for example, a stream that may recycle a bottoms mixture of the reactor 110 back into the reactor 110, or a stream may be included to release a gas from the reactor 110. Stream 111 may include at least apart of the reaction mixture.

In some embodiments, the carbonylation reaction in reactor 110 of system 100 may be performed in the presence of a catalyst. Catalysts may include, for example, rhodium catalysts and iridium catalysts.

Suitable rhodium catalysts are taught, for example, by U.S. Pat. No. 5,817,869, which is herein incorporated by reference. The rhodium catalysts may include rhodium metal and rhodium compounds. In an embodiment, the rhodium compounds may be selected from the group consisting of rhodium salts, rhodium oxides, rhodium acetates, organo-rhodium compounds, coordination compounds of rhodium, the like, and mixtures thereof in an embodiment, the rhodium compounds may be selected from the group consisting of Rh₂(CO)₄I₂, Rh₂(CO)₄Br₂, Rh₂(CO)₄Cl₂, Rh(CH₃CO₂)₂, Rh(CH₃CO₂)₃, [H]Rh(CO)₂I₂, the like, and mixtures thereof. In an embodiment, the rhodium compounds may be selected from the group consisting of [H]Rh(CO)₂I₂, Rh(CH₃CO₂)₂, the like, and mixtures thereof.

Suitable iridium catalysts are taught, for example, by U.S. Pat. No. 5,932,764. The iridium catalysts may include iridium metal and iridium compounds. Examples of suitable iridium compounds include IrCl₃, IrI₃, IrBr₃, [Ir(CO)₂I]₂, [Ir(CO)₂Cl]₂, [Ir(CO)₂Br]₂, [Ir(CO)₄I₂]—H+, [Ir(CO)₂Br₂]—H+, [IR(CO)₂I₂]—H+, [Ir(CH₃)I₃(CO)₂]—H+, Ir₄(CO)l₂, IrCl₃·4H₂O, IrBr₃·4H₂O, Ir₃(CO)l₂, Ir₂O₃, IrO₂, Ir(acac)(CO)₂, Ir(acac)₃, Ir(OAc)₃, [Ir₃O(OAc)₆(H₂O)₃][OAc], H₂[IrCl₆], the like, and mixtures thereof. In an embodiment, the iridium compounds may be selected from the group consisting of acetates, oxalates, acetoacetates, the like, and mixtures thereof. In an embodiment, the iridium compounds may be one or more acetates.

In an embodiment, the catalyst may be used with a co-catalyst. In an embodiment, co-catalysts may include metals and metal compounds selected from the group consisting of osmium, rhenium, ruthenium, cadmium, mercury, zinc, gallium, indium, and tungsten, their compounds, the like, and mixtures thereof. In an embodiment, co-catalysts may be selected from the group consisting of ruthenium compounds and osmium compounds. In an embodiment, co-catalysts may be one or more ruthenium compounds. In an embodiment, the co-catalysts may be one or more acetates.

The reaction rate depends upon the concentration of the catalyst in the reaction mixture in reactor 110. In an embodiment, the catalyst concentration may be in a range from about 1.0 mmol to about 100 mmol catalyst per liter (mmol/l) of reaction mixture. In some embodiments the catalyst concentration is at least 2.0 mmol/l, or at least 5.0 mmol/l, or at least 7.5 mmol/l. In some embodiments the catalyst concentration is at most 75 mmol/l, or at most 50 mmol/l, or at least 25 mmol/l. In particular embodiments, the catalyst concentration is from about 2.0 to about 75 mmol/l, or from about 5.0 to about 50 mmol/l, or from about 7.5 to about 25 mmol/l.

In an embodiment, the carbonylation reaction in reactor 110 of system 100 may be performed in the presence of a catalyst stabilizer. Suitable catalyst stabilizers include at least two types of catalyst stabilizers. The first type of catalyst stabilizer may be a metal iodide salt such as lithium iodide. The second type of catalyst stabilizer may be a non-salt stabilizer. In an embodiment, non-salt stabilizers may be pentavalent Group VA oxides, such as that disclosed in U.S. Pat. No. 9,790,159 which is herein incorporated by reference. In an embodiment, the catalyst stabilizer may be one or more phosphine oxides. In an embodiment, the catalyst may be CYTOP 503 from Solvay.

The one or more phosphine oxides, in one or more embodiments, are represented by the formula R₃PO, where R is alkyl or aryl, O is oxygen, P is phosphorous. In one or more embodiments, the one or more phosphine oxides include a compound mixture of at least four phosphine oxides, where each phosphine oxide has the formula OPX₃, wherein O is oxygen, P is phosphorous and X is independently selected from C₄-C₁₈ alkyls, C₄-C₁₈ aryls, C₄-C₁₈ cyclic alkyls, C₄-C₁₈ cyclic aryls and combinations thereof. Each phosphine oxide has at least 15, or at least 18 total carbon atoms.

Examples of suitable phosphine oxides for use in the compound mixture include, but are not limited to, tri-n-hexylphosphine oxide (THPO), tri-n-octylphosphine oxide (TOPO), tris(2,4,4-trimethylpentyl)-phosphine oxide, tricyclohexylphosphine oxide, tri-n-dodecylphosphine oxide, tri-n-octadecylphosphine oxide, tris(2-ethylhexyl)phosphine oxide, di-n-octylethylphosphine oxide, di-n-hexylisobutylphosphine oxide, octyldiisobutylphosphine oxide, tribenzylphosphine oxide, di-n-hexylbenzylphosphine oxide, di-n-octylbenzylphosphine oxide, 9-octyl-9-phosphabicyclo [3.3.1]nonane-9-oxide, dihexylmonooctylphosphine oxide, dioctylmonohexylphosphine oxide, dihexylmonodecylphosphine oxide, didecylmonohexylphosphine oxide, dioctylmonodecylphosphine oxide, didecylmonooctylphosphine oxide, and dihexylmonobutylphosphine oxide and the like.

The compound mixture includes from 1 wt % to 60 wt %, or from 35 wt % to 50 wt % of each phosphine oxide based on the total weight of compound mixture. In one or more specific, non-limiting embodiments, the compound mixture includes TOPO, THPO, dihexylmonooctylphosphine oxide and dioctylmonohexylphosphine oxide. For example, the compound mixture may include from 40 wt % to 44 wt % dioctylmonohexylphosphine oxide, from 28 wt % to 32 wt % dihexylmonooctylphosphine oxide, from 8 wt % to 16 wt % THPO and from 12 wt % to 16 wt % TOPO, for example.

In one or more embodiments, the compound mixture exhibits a melting point of less than 20° C., or less than 10° C., or less than 0° C., for example.

In one or more specific embodiments, the compound mixture is Cyanex™ 923, commercially available from Cytec Corporation.

The amount of pentavalent Group VA oxide, when used, is such that a ratio to rhodium is greater than about 60:1. In some embodiments, the ratio of the pentavalent Group 15 oxide to rhodium is from about 60:1 to about 500:1. In some embodiments, from about 0.1 to about 3 M of the pentavalent Group 15 oxide may be in the reaction mixture. In some embodiments, from about 0.15 to about 1.5 M, or from 0.25 to 1.2 M, of the pentavalent Group 15 oxide may be in the reaction mixture.

In other embodiments, the reaction may occur in the absence of a stabilizer selected from the group of metal iodide salts and non-metal stabilizers such as pentavalent Group 15 oxides. In further embodiments, the catalyst stabilizer may consist of a complexing agent which is brought into contact with the reaction mixture stream 111 in the flash vessel 120.

In an embodiment, hydrogen may also be fed into the reactor 110. Addition of hydrogen can enhance the carbonylation efficiency. In an embodiment, the concentration of hydrogen may be in a range of from about 0.1 mol % to about 5 mol % of carbon monoxide in the reactor 110. In an embodiment, the concentration of hydrogen may be in a range of from about 0.3 mol % to about 3 mol % of carbon monoxide in the reactor 110.

In an embodiment, the carbonylation reaction in reactor 110 of system 100 may be performed in the presence of water. In an embodiment, the concentration of water is from about 2 wt % to about 14 wt % based on the total weight of the reaction mixture. In an embodiment, the water concentration is from about 2 wt % to about 10 wt %. In an embodiment, the water concentration is from about 4 wt % to about 8 wt %.

In an embodiment, the carbonylation reaction may be performed in the presence of methyl acetate. Methyl acetate may be formed in situ. In embodiments, methyl acetate may be added as a starting material to the reaction mixture. In an embodiment, the concentration of methyl acetate may be from about 2 wt % to about 20 wt % based on the total weight of the reaction mixture. In an embodiment, the concentration of methyl acetate may be from about 2 wt % to about 16 wt %. In an embodiment, the concentration of methyl acetate may be from about 2 wt % to about 8 wt %. Alternatively, methyl acetate or a mixture of methyl acetate and methanol from byproduct streams of the methanolysis of polyvinyl acetate or ethylene-vinyl acetate copolymers can be used for the carbonylation reaction.

In an embodiment, the carbonylation reaction may be performed in the presence of methyl iodide. Methyl iodide may be a catalyst promoter. In an embodiment, the concentration of MeI may be from about 0.6 wt % to about 36 wt % based on the total weight of the reaction mixture. In an embodiment, the concentration of MeI may be from about 4 wt % to about 24 wt %. In an embodiment, the concentration of MeI may be from about 6 wt % to about 20 wt %. Alternatively, MeI may be generated in the reactor 110 by adding HI.

In an embodiment, methanol and carbon monoxide may be fed to the reactor 110 in stream 112 and stream 114, respectively. The methanol feed stream to the reactor 110 may come from a syngas-methanol facility or any other source. Methanol does not react directly with carbon monoxide to form acetic acid. It is converted to MeI by the HI present in the reactor 110 and then reacts with carbon monoxide and water to give acetic acid and regenerate the HI.

In an embodiment, the carbonylation reaction in reactor 110 of system 100 may occur at a temperature within the range of about 120° C. to about 250° C., alternatively, about 150° C. to about 250° C., alternatively, about 150° C. to about 200° C. In an embodiment, the carbonylation reaction in reactor 110 of system 100 may be performed under a pressure within the range of about 200 psia (1.4 MPa-a) to 2000 psia (13.8 MPa-a), alternatively, about 200 psia (1,379 kPa-a) to about 1,000 psia (6.8 MPa-a), alternatively, about 300 psia (2.1 MPa-a) to about 500 psia (3.4 MPa-a).

In some embodiments, the flash vessel 120 may be configured to receive stream 111 from the reactor 110. In the flash vessel 120, stream 111 may be separated into a vapor stream 126 and a liquid stream 121. The vapor stream 126 may be communicated to the light-ends column 130, and the liquid stream 121 may be communicated to the reactor 110 (stream 121 may thus be considered in the recycle area 108 and in the reactor area 102). In some embodiments, stream 126 may have acetic acid, water, methyl iodide, methyl acetate, HI, mixtures thereof and the like.

In an embodiment, the reaction mixture may be withdrawn from the reactor 110 through stream 111 and is flashed in flash vessel 120 to form a vapor stream 126 and a liquid stream 121. The reaction mixture in stream 111 may include acetic acid, methanol, methyl acetate, methyl iodide, carbon monoxide, carbon dioxide, water, HI, heavy impurities, catalyst, or combinations thereof. The flash vessel 120 may comprise any configuration for separating vapor and liquid components via a reduction in pressure. For example, the flash vessel 120 may comprise a flash tank, nozzle, valve, or combinations thereof.

The flash vessel 120 may have a pressure below that of the reactor 110. In an embodiment, the flash vessel 120 may have a pressure of from about 10 psig (69 kPa-g) to 100 psig (690 kPa-g). In an embodiment, the flash vessel 120 may have a temperature of from about 100° C. to 160° C.

The vapor stream 126 may include acetic acid and other volatile components such as methanol, methyl acetate, methyl iodide, carbon monoxide, carbon dioxide, water, entrained HI, complexed HI, and mixtures thereof. The liquid stream 121 may include the catalyst, complexed HI, HI, an azeotrope of HI and water, and mixtures thereof. The liquid stream 121 may further comprise sufficient amounts of water and acetic acid to carry and stabilize the catalyst, non-volatile catalyst stabilizers, or combinations thereof. The liquid stream 121 may recycle to the reactor 110. The vapor stream 126 may be communicated to light-ends column 130 for distillation.

The light-ends area 104 may include a separations column, for example a light-ends column 130, and associated equipment such as decanter 134, and streams associated with the light-ends column 130. For example, the light-ends area 104 may include light-ends column 130, a decanter 134, and streams 126, 131, 132, 133, 135, 136, 138, 139 and 160. The light-ends column is a fractioning or distillation column and includes equipment associated with the column, such as heat exchangers, decanters, pumps, compressors, valves, and the like.

In an embodiment, the light-ends column 130 may be a distillation column and associated equipment such as a decanter 134, pumps, compressors, valves, and other related equipment. The light-ends column 130 may be configured to receive stream 126 from the flash vessel 120. In the illustrated embodiment, stream 132 is the overhead product from the light-ends column 130, and stream 131 is bottoms product from the light-ends column 130. As indicated, light-ends column 130 may include a decanter 134, and stream 132 may pass into decanter 134.

Stream 135 may emit from decanter 134 and recycle back to the light-ends column 130. Heavy organic phase stream 138 may emit from decanter 134 and may recycle back to the reactor 110 via, for example, stream 112 or be combined with any of the other streams that feed the reactor (stream 138 may thus be considered in the recycle area 108, in the light-ends area 104, and in the reactor area 102). Light aqueous phase stream 139 may recycle a portion of the light phase of decanter 134 back to the reactor 110 via, for example, stream 112. Stream 136 may emit from the light-ends column 130. Other streams may be included such as, for example, a stream that may recycle a bottoms mixture of the light-ends column 130 back into the light-ends column 130. Streams received by or emitted from the light-ends column 130 may pass through a pump, compressor, heat exchanger, and the like as is common in the art.

In an embodiment, the vapor stream 126 may be distilled in a light-ends column 130 to form an overhead stream 132, a crude acetic acid product stream 136, and a bottom stream 131. In an embodiment, the light-ends column 130 may have at least 10 theoretical stages or 16 actual stages. In an alternative embodiment, the light-ends column 130 may have at least 14 theoretical stages. In an alternative embodiment, the light-ends column 130 may have at least 18 theoretical stages. In embodiments, one actual stage may equal approximately 0.6 theoretical stages. Actual stages can be trays or packing. The reaction mixture may be fed via stream 126 to the light-ends column 130 at the bottom or the first stage of the column 130.

Stream 132 may include acetaldehyde, water, carbon monoxide, carbon dioxide, methyl iodide, methyl acetate, methanol and acetic acid, and mixtures thereof. Stream 131 may have acetic acid, methyl iodide, methyl acetate, HI, water, and mixtures thereof. Stream 136 may have acetic acid, HI, water, heavy impurities, and mixtures thereof.

In an embodiment, the light-ends column 130 may be operated at an overhead pressure within the range of 20 psia (138 kPa-a) to 40 psia (276 kPa-a), alternatively, the overhead pressure may be within the range of 30 psia (207 kPa-a) to 35 psia (241 kPa-a). In an embodiment, the overhead temperature may be within the range of 95° C. to 135° C., alternatively, the overhead temperature may be within the range of 110° C. to 135° C., alternatively, the overhead temperature may be within the range of 125° C. to 135° C. In an embodiment, the light-ends column 130 may be operated at a bottom pressure within the range of 25 psia (172 kPa-a) to 45 psia (310 kPa-a), alternatively, the bottom pressure may be within the range of 30 psia (207 kPa-a) to 40 psia (276 kPa-a).

The overhead stream 132 from the light-ends column 130 may be condensed and separated in a decanter 134 to form a light aqueous phase and a heavy organic phase. In some embodiments, a portion or all of the heavy organic phase may be sent as stream 138 for further processing, as discussed below. In some embodiments, a portion or all of the light aqueous phase may be sent as stream 139 for further processing, as discussed below. In some embodiments, a portion or all of the heavy organic phase may be sent as stream 138 and a portion or all of the light aqueous phase may be sent as stream 139 for further processing, as discussed below. Further, a portion of stream 138 and/or stream 139 may be optionally recycled to the reactor 110 via stream 112, for example. It should be noted that the portion of streams 138 and/or 139 sent to adsorption bed 200 via streams 138a and/or 139a, respectively, for treatment to remove acetaldehyde and the other portion of the streams 138 and/or 139, respectively, recycled to the reactor 110 may each originate as independent streams from the decanter 134 heavy organic phase and/or light aqueous phase, respectively. In some embodiments, the light aqueous phase from the decanter 134 may be recycled to the light-ends column 130 in stream 135 or may be recycled to the reactor 110 in stream 139 via stream 112, for example.

The heavy organic phase stream 138 may have acetaldehyde, MeI, methyl acetate, acetic acid, water, and mixtures thereof. In an embodiment, stream 138 may be essentially non-aqueous with a water concentration of less than 1 wt %. In an embodiment, stream 138 may have MeI greater than 50% by weight of the stream. The light aqueous phase in streams 135 and 139 may have water (greater than 50% by weight of the stream), acetic acid, methyl acetate, methyl iodide, acetaldehyde, and mixtures thereof. Make-up water may be introduced into the decanter 134 via an external source 133.

In an embodiment, the bottom temperature of the light-ends column 130 may be within the range of 115° C. to 155° C., alternatively, the bottom temperature is within the range of 125° C. to 135° C. In an embodiment, crude acetic acid in stream 136 may be emitted from the light-ends column 130 as a liquid side-draw. Stream 136 may be operated at a pressure within the range of 25 psia (172 kPa-a) to 45 psia (310 kPa-a), alternatively, the pressure may be within the range of 30 psia (207 kPa-a) to 40 psia (276 kPa-a). In an embodiment, the temperature of stream 136 may be within the range of 110° C. to 140° C., alternatively, the temperature may be within the range of 125° C. to 135° C. Stream 136 may be taken between the fifth to the eighth actual stage of the light-ends column 130.

The purification area 106 may include a drying column 140, optionally a heavy-ends column 150, and associated equipment, and streams associated with the drying column 140 and heavy-ends column 150. For example, the purification area 106 may include drying column 140, heavy-ends column 150 and streams 136, 141, 142, 145, 148, 151, 152 and 156. The heavy-ends column is a fractioning or distillation column and includes any equipment associated with the column, such as heat exchangers, decanters, pumps, compressors, valves, and the like.

In an embodiment, the drying column 140 may be a vessel and associated equipment such as heat exchangers, decanters, pumps, compressors, valves, and the like. The drying column 140 may be configured to receive stream 136 from the light-ends column 130. The drying column 140 may separate components of stream 136 into streams 142 and 141.

Stream 142 may emit from the drying column 140, recycle back to the drying column via stream 145, and/or recycle back to the reactor 110 through stream 148 (via, for example, stream 112). Stream 141 may emit from the drying column 140 and may include de-watered crude acetic acid product. Stream 142 may pass through equipment such as, for example, a heat exchanger or separation vessel before streams 145 or 148 recycle components of stream 142. Other streams may be included such as, for example, a stream may recycle a bottoms mixture of the drying column 140 back into the drying column 140. Streams received by or emitted from the drying column 140 may pass through a pump, compressor, heat exchanger, separation vessel, and the like as is common in the art.

The heavy-ends column 150 may be a distillation column and associated equipment such as heat exchangers, decanters, pumps, compressors, valves, and the like. The heavy-ends column 150 may be configured to receive stream 141 from the drying column 140. The heavy-ends column 150 may separate components from stream 141 into streams 151, 152, and 156. Streams 151 and 152 may be sent to additional processing equipment (not shown) for further processing. Stream 152 may also be recycled, for example, to light-ends column 130. Stream 156 may include acetic acid product.

In one or more embodiments, the crude acetic acid in stream 136 may be optionally subjected to further purification, such as, but not limited to, drying-distillation, in drying column 140 to remove water and heavy-ends distillation in stream 141. Stream 141 may be communicated to heavy-ends column 150 where heavy impurities such as propionic acid may be removed in stream 151 and final acetic acid product may be recovered in stream 156.

The purification area 106 may further include an adsorption bed 200. Selected streams may be passed therethrough prior to proceeding downstream. For example, heavy organic phase stream 138 may be passed through the adsorption bed 200 via stream 138a to form stream 138b. Alternatively, or combination therewith, light aqueous phase stream 139 may be passed through the adsorption bed 200 via stream 139a to form stream 139b.

The recycle area 108 may include process streams recycled to the reaction area 102 and/or light-ends area 104. For example, in FIG. 1, the recycle area 108 may include streams 121, 138, 139 and 148. The recycle streams may be products or compositions recovered from a processing step downstream of the flash vessel 120 and which is recycled to the reactor 110, flash vessel 120, or light-ends column 130, and so forth.

Alternative embodiments for the carboxylic acid production system 100 may be found in U.S. Pat. No. 6,552,221, which is incorporated herein by reference.

In some embodiments, a method for removing acetaldehyde from an acetic acid system, comprises providing from the acetic acid system a solution, comprising acetic acid, water, methyl acetate, methyl iodide, and acetaldehyde, wherein the acetaldehyde is present in a first concentration based on the total weight of the solution. The method further comprises contacting the solution with a phyllosilicate clay-based adsorbent at adsorption conditions sufficient to produce a treated solution, wherein: the solution has a first acetaldehyde content; the treated solution has a second acetaldehyde content; and the second acetaldehyde content is less than the first acetaldehyde content. In some embodiments, the ratio of the first acetaldehyde content to the second acetaldehyde content is greater than or equal to 2.0, greater than or equal to 2.5, greater than or equal to 4.0, or greater than or equal to 10.0.

In some embodiments of the method, the phyllosilicate clay-based adsorbent comprises a kaolinite, a smectite, a vermiculite, an illite, a chlorite, or a combination thereof. In some embodiments of the method, the phyllosilicate clay-based adsorbent comprises a smectite. In some embodiments of the method, the smectite comprises a montmorillonite, beidellite, nantronite, saponite, hectorite, a bentonite, or a combination thereof. In some embodiments of the method, the smectite comprises a montmorillonite, a bentonite, or a combination thereof.

In some embodiments of the method, the phyllosilicate clay-based adsorbent comprises interlayer cations, interlayer metal-oxide pillars, or a combination thereof.

In some embodiments of the method, the phyllosilicate clay-based adsorbent is acidified.

In some embodiments of the method, the phyllosilicate clay-based adsorbent has a porosity as measured by an average pore volume in the range of from 0.1 to 1.4 cm³/g, from 0.2 to 1.2 cm³/g, or from 0.3 to 1.0 cm³/g.

In some embodiments of the method, the phyllosilicate clay-based adsorbent has a surface area in the range of from 50 m²/g to 800 m²/g, from 100 m²/g to 750 m²/g, from 150 m²/g to 700 m²/g, or from 200 m²/g to 600 m²/g.

In some embodiments of the method, the phyllosilicate clay-based adsorbent has an average removal capability of greater than or equal to 0.3 g acetaldehyde/g of adsorbent, greater than or equal to 0.4 g acetaldehyde/g of adsorbent, greater than or equal to 0.5 g acetaldehyde/g of adsorbent, or greater than or equal to 0.6 g acetaldehyde/g of adsorbent.

In some embodiments of the method, a concentration of acetaldehyde in the solution is selectively reduced by an amount greater than or equal to 50%, greater than or equal to 60%, greater than or equal to 70%, greater than or equal to 80%, greater than or equal to 85%, greater than or equal to 90%, greater than or equal to 95%, or greater than or equal to 98%.

In some embodiments of the method, the phyllosilicate clay-based adsorbent is disposed on a fixed bed. In further embodiments, the phyllosilicate clay-based adsorbent is loaded in the fixed bed at a level sufficient to provide a loading in a range of from 1 g to 10 g phyllosilicate clay-based adsorbent/g acetaldehyde to be adsorbed, from 2 g to 8 g phyllosilicate clay-based adsorbent/g acetaldehyde to be adsorbed, or from 4 g to 6 g phyllosilicate clay-based adsorbent/g acetaldehyde to be adsorbed.

In some embodiments of the method, the adsorption conditions comprise an adsorption temperature in a range of from room temperature to 250° C., from room temperature to 225° C., or from room temperature to 200° C.

In some embodiments of the method, the phyllosilicate clay-based adsorbent is capable of undergoing regeneration.

### Certain Embodiments

In some aspects, processes for the production of acetic acid are disclosed. In an embodiment, a process for the production of acetic acid comprises reacting methanol and carbon monoxide in the presence of a carbonylation catalyst to form acetic acid in an acetic acid production reactor. The process further comprises flashing a reaction mixture discharged from the acetic acid production reactor into a vapor stream and a liquid stream, wherein the vapor stream comprises acetic acid, water, methanol, methyl acetate, methyl iodide, and acetaldehyde. The vapor stream is separated by distillation in a distillation column into: a product side stream comprising acetic acid and water; a bottoms stream; and an overhead stream comprising methyl iodide, water, methyl acetate, acetic acid, and acetaldehyde. The overhead stream in condensed into: a light aqueous phase stream, comprising methyl iodide, acetaldehyde, water, methyl acetate, and acetic acid; and a heavy organic phase stream, comprising methyl iodide, acetaldehyde, water, methyl acetate, and acetic acid. An intermediate process stream with a phyllosilicate clay-based adsorbent at adsorption conditions sufficient to produce a treated intermediate process stream, wherein the intermediate process stream comprises at least a portion of the light aqueous phase stream, at least a portion of the heavy organic phase stream, or a combination thereof. The intermediate process stream has a first acetaldehyde content, the treated intermediate process stream has a second acetaldehyde content, and the second acetaldehyde content is less than the first acetaldehyde content.

In some embodiments, in addition to the foregoing steps of the process for producing acetic acid, the process can be further characterized by one of more of the following:
a) the ratio of the first acetaldehyde content to the second acetaldehyde content is greater than or equal to 2.0, greater than or equal to 2.5, greater than or equal to 4.0, or greater than or equal to 10.0;
b) the process further comprises recycling the treated intermediate process stream to the acetic acid production reactor;
c) the phyllosilicate clay-based adsorbent comprises a kaolinite, a smectite, a vermiculite, an illite, a chlorite, or a combination thereof, or a smectite, wherein in further embodiments, the smectite comprises a montmorillonite, beidellite, nantronite, saponite, hectorite, a bentonite, or a combination thereof, or a montmorillonite, a bentonite, or a combination thereof;
d) the phyllosilicate clay-based adsorbent comprises interlayer cations, interlayer metal-oxide pillars, or a combination thereof;
e) the phyllosilicate clay-based adsorbent is acidified;
f) the phyllosilicate clay-based adsorbent has a porosity as measured by an average pore volume in the range of from 0.1 to 1.4 cm³/g, from 0.2 to 1.2 cm³/g, or from 0.3 to 1.0 m³/g;
g) the phyllosilicate clay-based adsorbent has a surface area in the range of from 50 m²/g to 800 m²/g, from 100 m²/g to 750 m²/g, from 150 m²/g to 700 m²/g, or from 200 m²/g to 600 m²/g;
h) the phyllosilicate clay-based adsorbent has an average removal capability of greater than or equal to 0.3 g acetaldehyde/g of adsorbent, greater than or equal to 0.4 g acetaldehyde/g of adsorbent, greater than or equal to 0.5 g acetaldehyde/g of adsorbent, or greater than or equal to 0.6 g acetaldehyde/g of adsorbent.
i) a concentration of acetaldehyde in the intermediate product stream is selectively reduced by an amount greater than or equal to 50%, greater than or equal to 60%, greater than or equal to 70%, greater than or equal to 80%, greater than or equal to 85%, greater than or equal to 90%, greater than or equal to 95%, or greater than or equal to 98%;
j) the phyllosilicate clay-based adsorbent is disposed on a fixed bed, wherein in further embodiments, the phyllosilicate clay-based adsorbent is loaded in the fixed bed at a level sufficient to provide a loading in a range of from 1 g to 10 g phyllosilicate clay-based adsorbent/g acetaldehyde to be adsorbed, from 2 g to 8 g phyllosilicate clay-based adsorbent/g acetaldehyde to be adsorbed, or from 4 g to 6 g phyllosilicate clay-based adsorbent/g acetaldehyde to be adsorbed;
k) the adsorption conditions comprise an adsorption temperature in a range of from room temperature to 250° C., from room temperature to 225° C., or from room temperature to 200° C.;
l) the light aqueous phase stream comprises greater than 20 wt % water and the heavy organic phase stream comprises less than 1 wt % water; and
m) phyllosilicate clay-based adsorbent is capable of undergoing regeneration.

In some aspects, methods for removing acetaldehyde are disclosed. In an embodiment, a method for removing acetaldehyde from an acetic acid system, comprises providing from the acetic acid system a solution, comprising acetic acid, water, methyl acetate, methyl iodide, and acetaldehyde, wherein the acetaldehyde is present in a first concentration based on the total weight of the solution. The solution is contacted with a phyllosilicate clay-based adsorbent at adsorption conditions sufficient to produce a treated solution, wherein the solution has a first acetaldehyde content, the treated solution has a second acetaldehyde content, and the second acetaldehyde content is less than the first acetaldehyde content.
a) the ratio of the first acetaldehyde content to the second acetaldehyde content is greater than or equal to 2.0, greater than or equal to 2.5, greater than or equal to 4.0, or greater than or equal to 10.0;
b) the phyllosilicate clay-based adsorbent comprises a kaolinite, a smectite, a vermiculite, an illite, a chlorite, or a combination thereof, or a smectite, wherein in further embodiments, the smectite comprises a montmorillonite, beidellite, nantronite, saponite, hectorite, a bentonite, or a combination thereof, or a montmorillonite, a bentonite, or a combination thereof;
c) the phyllosilicate clay-based adsorbent comprises interlayer cations, interlayer metal-oxide pillars, or a combination thereof;
d) the phyllosilicate clay-based adsorbent is acidified;
e) the phyllosilicate clay-based adsorbent has a porosity as measured by an average pore volume in the range of from 0.1 to 1.4 cm³/g, from 0.2 to 1.2 cm³/g, or from 0.3 to 1.0 m³/g;
f) the phyllosilicate clay-based adsorbent has a surface area in the range of from 50 m²/g to 800 m²/g, from 100 m²/g to 750 m²/g, from 150 m²/g to 700 m²/g, or from 200 m²/g to 600 m²/g;
g) the phyllosilicate clay-based adsorbent has an average removal capability of greater than or equal to 0.3 g acetaldehyde/g of adsorbent, greater than or equal to 0.4 g acetaldehyde/g of adsorbent, greater than or equal to 0.5 g acetaldehyde/g of adsorbent, or greater than or equal to 0.6 g acetaldehyde/g of adsorbent.
h) a concentration of acetaldehyde in the intermediate product stream is selectively reduced by an amount greater than or equal to 50%, greater than or equal to 60%, greater than or equal to 70%, greater than or equal to 80%, greater than or equal to 85%, greater than or equal to 90%, greater than or equal to 95%, or greater than or equal to 98%;
i) the phyllosilicate clay-based adsorbent is disposed on a fixed bed, wherein in further embodiments, the phyllosilicate clay-based adsorbent is loaded in the fixed bed at a level sufficient to provide a loading in a range of from 1 g to 10 g phyllosilicate clay-based adsorbent/g acetaldehyde to be adsorbed, from 2 g to 8 g phyllosilicate clay-based adsorbent/g acetaldehyde to be adsorbed, or from 4 g to 6 g phyllosilicate clay-based adsorbent/g acetaldehyde to be adsorbed;
j) the adsorption conditions comprise an adsorption temperature in a range of from room temperature to 250° C., from room temperature to 225° C., or from room temperature to 200° C.;
k) phyllosilicate clay-based adsorbent is capable of undergoing regeneration.

The presently disclosed processes and methods are exemplified with respect to the examples below. These examples are included to demonstrate embodiments of the appended claims. However, these are exemplary only, and the invention can be broadly applied to any acetic acid production process. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### Examples

The following investigations and examples are intended to be illustrative only, and are not intended to be, nor should they be construed as, limiting the scope of the present invention in any way.

### Clays Tested

In the following examples, experiments were performed using clays having a general structure comprising various arrangements of tetrahedral and/or octahedral sheets of silica and/or alumina and interlayer spaces occupied by metal cations such as Na⁺ and by H₂O. In some experiments, the interlayer spacing was modified by a technique called pillaring in which a poly oxo metal cation is introduced and acts as a pillar between layers, forcing them apart. A wide range of interlayer spacings can was achieved by varying the metal in the pillaring agent.

Pillared clays were synthesized by mixing bentonite or montmorillonite clays with the pillaring reagent stirred solutions at room temperature. The mixture was then allowed to stand for several hours to ensure intercalation of the pillaring reagents into the interlayer spaces of the clay. The expanded interlayer spacing of pillared AQGS clays creates a porous structure that can trap and adsorb molecules from a solution. Pillaring creates a more stable structure with increased surface area and porosity.

Other clay variants tested included drying commercial clays and acidifying both pillared and non-pillared clays. Additional information regarding synthesis of pillared clays can be found in: Synthesis of Pillared Clay Catalyst for the Production of Hydrocarbon Fuel from Plastic Waste, Proceedings of the International Conference on Advances in Chemical Engineering, 2020; Synthesis and Application of Pillared Clay Heterogeneous Catalysts for Wastewater Treatment: A Review, RSC Advances, 8, 5197-5211, 2018; and Synthesis and Characterization of Al-Pillared Interlayered Bentonites, G.U Journal of Science, 21, 21-31, 2007; the contents of which are fully incorporated by reference herein.

### Starting Materials

Starting materials for the examples below are shown in Table 1.

TABLE 1

| | | Commercial | |
|---|---|---|---|
| Label | Composition | Identifier | Available from |
| MMT1 | Al Pillared Montmorillonite, SA = 250-280 m²/g, | Cat# 69907 | Sigma Aldrich, |
| | pH = 4.5, CEC = 0.13 meq/g, pore vol.¹ = 0.246 | | Burlington, MA |
| | cm³/g, pore size¹ = 3.9 nm | | |
| MMT2 | K10 Layered Montmorillonite, SA = 240 m²/g, | Cat# 281522 | Sigma Aldrich, |
| | pH = 3.2, CEC = 0.36 meq/g, pore vol. = 0.28 | | Burlington, MA |
| | cm³/g (0-80 nm), 0.25 cm³/g (0-24 nm), 0.19 | | |
| | cm³/g (0-14 nm) | | |
| MMT3 | K30 Layered Montmorillonite, SA = 330 m²/g, | Cat# 69904 | Sigma Aldrich, |
| | pH = 3.1, CEC = 0.5 meq/g, pore vol. = vol. = 0.50 | | Burlington, MA |
| | cm³/g (0-80 nm), 0.44 cm³/g (0-24 nm), 0.38 | | |
| | cm³/g (0-14 nm) | | |
| BNT1 | 200-mesh dry powder Wyoming sodium | Aquagel Gold | Baroid Fluid Technologies, |
| | bentonite that contains no polymer additive, | Seal ™ Bentonite | Houston, TX |
| | pH 8-10 | (AQGS) | |
| SAPO1 | SAPO-34 | Cat# MSSA3421 | ACS Material, LLC, |
| | | | Pasadena, CA |
| A15 | Amberlyst ™ 15 | Cat# 216380 | Sigma Aldrich, |
| | | | Burlington, MA |

¹Pore volume and size determined by Barrett-Joyner-Halenda (BJH) model

### Synthesized Materials

Experimental materials synthesized from starting material for the examples below are shown in Table 2, below.

### Preparation of Al Pillared Clay

66 mLs of 0.4M Al(NO₃)₃ solution was added dropwise over 2 hours to a stirred solution of 134 mLs of 0.4M NaOH at room temperature. The solution was then transferred to a sample bottle and allowed to age for 3 days.

25 mLs of the pillaring solution was added to a 50 mL round bottom flask. 10 mLs H₂O was added and vigorous stirring at room temperature was started. While being stirred, 2.5 g of clay was slowly added. After 4 hours of further stirring, the mixture was still in slurry form rather than sludge.

The slurry was filtered slowly on a medium frit to a paste rather than a cake. The paste was washed with 30 mLs of H₂O. The frit containing the paste was then placed in furnace at 130° C. to dry for 3 hours. The dried material was ground with mortar and pestle and placed in a sample vial.

### Preparation of Ce Pillared Clay

66 mLs of 0.4M Ce(NO₃)₃ solution was added dropwise over 2 hours to a stirred solution of 134 mLs of 0.4M NaOH at room temperature. The solution was then transferred to a sample bottle and allowed to age for 4 days.

50 mLs of the pillaring solution was added to 250 mL boiling flask and stirred vigorously at room temperature. 5 g of clay was added slowly, allowing the clay to mix well. After 4 hours of further stirring, the mixture was still in slurry form rather than sludge.

The slurry filtered slowly on a medium frit to a paste rather than a cake. The paste was washed with 30 mLs of H₂O. The frit containing the paste was placed in furnace at 130° C. to dry for 3 hours. The dried material was ground with mortar and pestle and placed in a sample vial.

### Preparation of Al/Fe Pillared Clay

33 mLs each of 0.4M Al(NO₃)₃ and FeNO₃ solutions were combined in in a sample bottle. 134 mLs of 0.4M NaOH was added to a 250 mLs boiling flask and stirred vigorously at room temperature while the Al/Fe solution was added dropwise over 4 hours. The solution was allowed to age for 5 days.

25 mLs of the pillaring solution was added to a 50 mL round bottom flask. 10 mLs H₂O was added and vigorous stirring at room temperature was started. While being stirred, 2.5 g of clay was slowly added. After 4 hours of further stirring, the mixture was still in slurry form rather than sludge.

The slurry was filtered slowly on a medium frit to a paste rather than a cake. The paste was washed with 30 mLs of H₂O. The frit containing the paste was then placed in furnace at 130° C. to dry for 3 hours. The dried material was ground with mortar and pestle and placed in a sample vial.

### Preparation of Ga Pillared Clay

66 mLs of 0.4M Ga(NO₃)₃ solution was added dropwise over 2 hours to a stirred solution of 134 mLs of 0.4M NaOH at room temperature. The solution was then transferred to a sample bottle and allowed to age for 4 days.

50 mLs of the above solution was combined with 50 mLs of H₂O in a 250 mL boiling flask. This solution was stirred vigorously at room temperature while 5 g of clay was slowly added. After clay addition was complete, the slurry was stirred for a further 4 hours at room temperature at which point it had transformed from a clumpy slurry to a sludge.

The resulting sludgy mixture was poured onto a medium frit and filtered under vacuum. The material on frit was washed once with 30 mLs of H₂O. Even with vacuum filtration, the remaining material on the frit was a sludge rather than a cake. Using a spatula, the sludge was transferred to centrifuge tubes and the tubes were centrifuged at 4,000 rpm for 10 minutes. The liquid was decanted off, H₂O added and then a spatula was used to stir up solid at bottom that was coated to sides of tube in order to optimize contact of solid with water wash. After centrifugation, a second similar washing procedure was carried out.

The polypropylene centrifuge tubes were placed in a beaker and put in furnace for 4 hours at 115° C. The partially dried material (now a paste) was then scraped out of the tubes, added to porcelain dish, and returned to a furnace set at 130° C. to complete drying. Dried material was ground with mortar and pestle and placed in a sample vial.

### Preparation of Zr Pillared Clay

The bulk pillaring solution was prepared as described above except that ZrOCl₂ was used. It was aged for 5 days before being used.

25 mLs of the pillaring solution was added to a 50 mL round bottom flask. 10 mLs H₂O was added and vigorous stirring at room temperature was started. While being stirred, 2.5 g of clay was slowly added. After 4 hours of further stirring, the mixture was still in slurry form rather than sludge.

The slurry was filtered slowly on a medium frit to a paste rather than a cake. The paste was washed with 30 mLs of H₂O. The frit containing the paste was then placed in furnace at 130° C. to dry for 3 hours. The dried material was ground with mortar and pestle and placed in a sample vial.

### Acidification of Pillared Clay

This procedure applies to all pillared materials.

1.2 g of dried clay was added to 25 mL round bottom flask with stirrer. 10 mLs of 0.5M H₂SO₄ was added to the flask and stirred vigorously for 90 minutes at room temperature. Even with vigorous stirring, some clumping occurred.

After 90 minutes of stirring, the mixtures could best be categorized as sludges rather than slurries. The sludge material was then poured onto a medium frit and filtered slowly under vacuum. The remaining sludge in the flask was contacted with 20 mLs H₂O, then shaken to get the sludge suspended in the water. The mixture was then poured onto frit. The resulting paste on the frit is washed with 10 mLs of H₂O slowly on a medium frit. The frit was then dried in a furnace at 130° C. for 2 hours, after which the material was ground with mortar and pestle and transferred to a sample vial.

TABLE 2

| Label | Composition |
|---|---|
| MMT1d | Dried MMT1 |
| MMT1a | Acidified MMT1 |
| MMT3d | Dried MMT3 |
| BNT1d | Dried BNT1 |
| BNT1p1 | Al Pillared BNT1 |
| BNT1p2 | Ce Pillared BNT1 |
| BNT1p3 | Al/Fe Pillared BNT1 |
| BNT1p4 | Ga Pillared BNT1 |
| BNT1p5 | Zr Pillared BNT1 |
| SAPO1d | Dried SAPO1 |

### Experimental Methods

All experiments to measure the adsorption performance of the various clays and comparative materials were carried out in stirred vials at room temperature (˜24° C.). Each experiment was based on a small volume of clay (ranging from 0.075 g to 0.3 g) clay slurried with 1 ml to 3 ml of acetaldehyde stock solution in octane, decane, or H₂O. Stock solution concentration of acetaldehyde in water was 14%, based on total weight of acetaldehyde and water. Clay was added to the aqueous solution in a ratio of 0.3 g clay/3 mL solution. Stock solution concentration of acetaldehyde in octane or decane was 4%, based on total weight of acetaldehyde and solvent. Clay was added to the organic solution in a ratio of 0.075 g clay/1 mL solution. Octane and decane are considered interchange due to chemical similarity. These concentrations were used to give sufficiently intense Fourier transform infrared spectroscopy (FTIR) absorbances to allow quantitative determination of acetaldehyde removal from the solution.

In all experiments, clay was first weighed into the vial. A magnetic stir bar was then added and stirring of the vial was commenced. The appropriate volume of acetaldehyde stock solution was then syringed into the vial. The vial was then sealed with a cap and allowed to stir for various times (as detailed in the examples) at room temperature. Studies indicated that any adsorption that was going to take place had largely occurred after 2 minutes and had completely finished after 15 minutes. Therefore, all capacity data reported in tables below are associated with 15 minute time points. The ratio of clay to stock solution in all experiments was set at 1 g of clay per gram of acetaldehyde in the aliquot of stock solution.

### Results

The majority of the studies described in this memo were carried out using a stock solution of acetaldehyde in octane. Octane was used as a surrogate for MeI in order to avoid the difficulties of frequent handling of MeI. As they are both organic, relatively non-polar, H₂O immiscible solvents, it was anticipated that they would be interchangeable with regard to any impact on adsorption or reaction of acetaldehyde. This octane/acetaldehyde stock solution was used as an approximate surrogate for the “decanter heavy organic phase stream,” as described above.

In 9 of the 13 clay samples that were studied with acetaldehyde/octane solutions, acetaldehyde adsorption appeared to take place with no evidence of catalytic formation of other species, such as paraldehyde (PA) and/or crotonaldehyde (CA). For the remaining 4 clay samples, acetaldehyde (HAc) disappearance from solution was accompanied by some formation of paraldehyde.

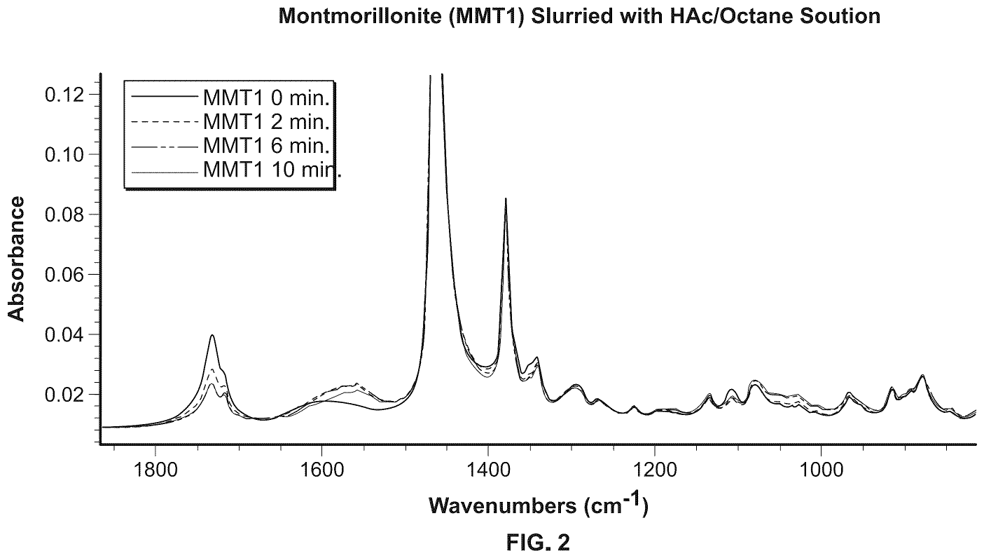

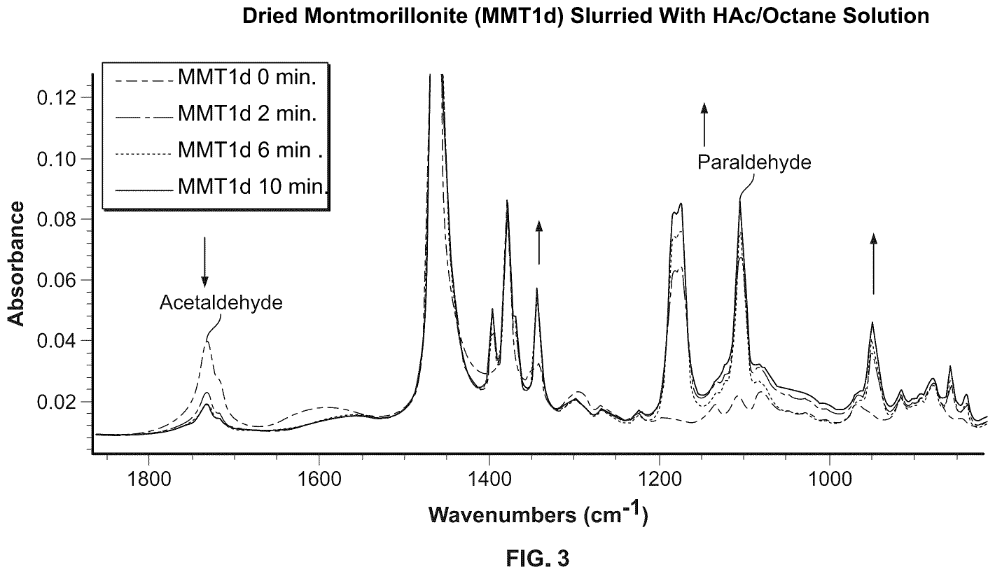

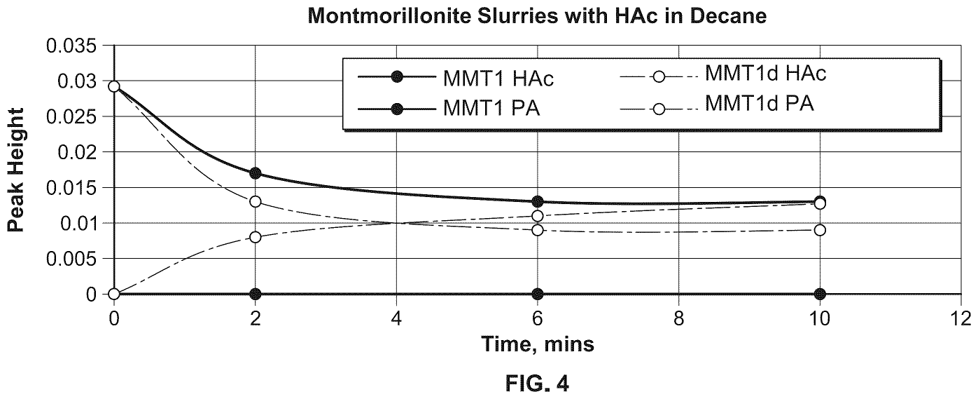

This is illustrated in the overlaid FTIR spectra in FIG. 2 and FIG. 3 for slurries of commercial Al pillared montmorillonite (MMT1) and dried montmorillonite (MMT1d), respectively. For the undried material, the decrease in absorbance of the acetaldehyde C═O peak at about 1740 cm⁻¹ is not accompanied by appearance of any new peaks in solution, as shown in FIG. 2. In contrast, for the dried material, multiple peaks corresponding with paraldehyde now appear in solution, as shown in FIG. 3. This assignment is based on a benchmark FTIR spectrum of a solution of commercial paraldehyde. The time profiles for reaction of montmorillonite and dried montmorillonite as represented in FIG. 2 and FIG. 3 are shown in FIG. 4. Related quantitative data are shown in Table 2.

TABLE 2

| | Peak Height | | | |
|---|---|---|---|---|
| Time | Montmorillonite | | Dried Montmorillonite | |
| (min.) | HAc (1733 cm⁻¹) | PA (856 cm⁻¹) | HAc (1733 cm⁻¹) | PA (856 cm⁻¹) |
| 0 | 0.0292 | 0 | 0.0292 | 0 |
| 2 | 0.017 | 0 | 0.013 | 0.008 |
| 6 | 0.013 | 0 | 0.009 | 0.011 |
| 10 | 0.013 | 0 | 0.009 | 0.0127 |

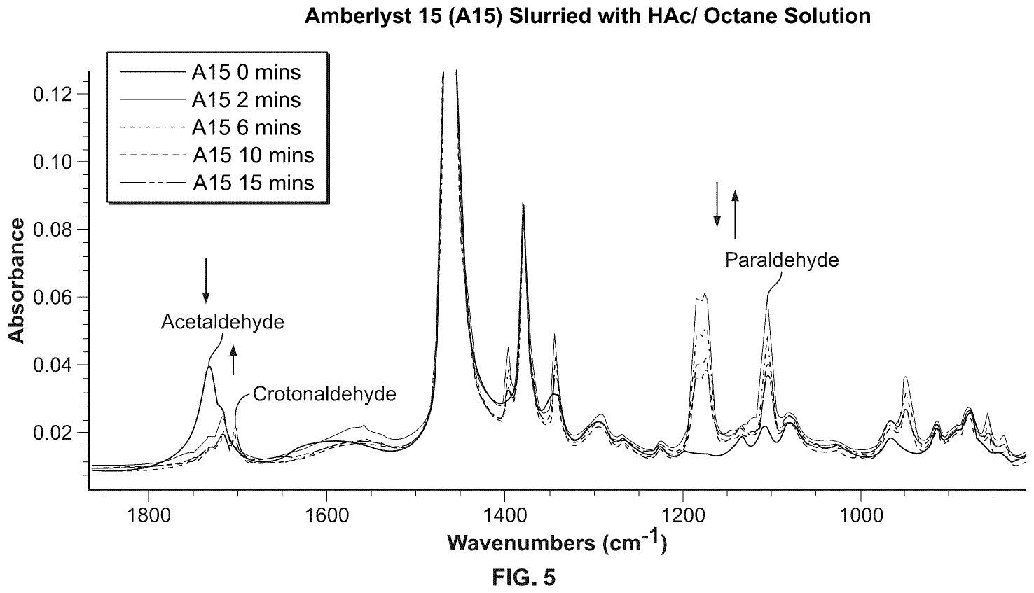

For reference purposes, the experiments were performed using Amberlyst™ 15 (a solid acid catalyst, available from DuPont Chemical Company, Pasadena, TX) was also carried out. The FTIR spectroscopic profile of the reaction is shown in FIG. 5. The expected behavior occurred with this strong acid resin, with initial and rapid kinetically controlled formation of paraldehyde followed by formation of the more thermodynamically stable crotonaldehyde.

Without wishing to be bound by any particular theory, it is believed that the higher adsorption of acetaldehyde demonstrated by montmorillonite as compared to dried montmorillonite results from montmorillonite (before drying) contains enough H₂O to prevent acetaldehyde oligomerization to paraldehyde. In some embodiments, “enough” H₂O is in the range of from 1 wt % to 13 wt %, from 3 wt % to 12 wt %, from 5 wt % to 11 wt %, or from 7 wt % to 10 wt %. It is believed that the absence of H₂O dried montmorillonite permits formation of paraldehyde, which is an unfavorable compound in the context of the process disclosed herein. Paraldehyde, a dimer of acetaldehyde, undergoes thermal degradation back to acetaldehyde at or above 60° C. back. Additionally, there is also potential for unwanted polymerization of the paraldehyde. As H₂O content of potential adsorbent may play a role, in several cases the adsorbents were dried in a furnace at 125° C. for 3 hours and the mass loss was quantified as shown in Table 3. It is a reasonable assumption that the mass losses are largely or solely associated with water loss.

TABLE 3

| Clay | % Mass Loss |
|---|---|
| SAPO1 | 19.5 |
| BNT1 | 9.4 |
| MMT3 | 8.4 |
| MMT1 | 7.8 |

The complete list of clays tested in HAc/octane solutions and their corresponding acetaldehyde removal performance is shown in Table 4. For comparison purposes, new data were also obtained for a silicoaluminophosphate, SAPO-34, whose removal capabilities had previously been studied in HAc/MeI solutions.

TABLE 4

| | | HAc | | | HAc removed |
|---|---|---|---|---|---|
| Ex. | Clay | Adsorption | PA | CA | (g/g adsorbent) |
| 1 | MMT1 | Yes | No | No | 0.55 |
| 2 | MMT1d | Yes* | Yes | No | 0.69 |
| 3 | MMT1a | Yes | No | No | 0.55 |
| 4 | MMT2 | Yes | No | No | 0.42 |
| 5 | MMT3 | Yes* | Yes | No | 0.45 |
| 6 | MMT3d | Yes* | Yes | No | 0.83 |
| 7 | BNT1 | Yes | No | No | 0.59 |
| 8 | BNT1d | Yes | No | No | 0.38 |
| 9 | BNT1p1 | Yes | No | No | 0.32 |
| 10 | BNT1p2 | Yes | No | No | 0.42 |
| 11 | BNT1p3 | Yes | No | No | 0.42 |
| 12 | BNT1p4 | Yes | No | No | 0.42 |
| 13 | BNT1p5 | Yes* | Yes | No | 0.35 |
| 14 | SAPO1 | Yes | No | No | 0.53 |
| 15 | SAPO1d | Yes | No | No | 0.35 |

*HAc adsorption in combination with some formation of PA

The tabular data indicate that in the cases of straightforward adsorption in Examples 1, 3, 4, 7-12, 14, and 15, an encouragingly high approximate 0.5 g acetaldehyde per g of clay is removed. Examples 2, 5, 6, and 13 are believed to demonstrate disappearance of HAc, spectroscopic analysis was not sufficiently accurate to confirm whether HAc disappearance was due to absorption of HAc, formation of PA, or a combination thereof. Comparative Examples 14 and 15 using SAPO-34, show that the clay materials disclosed herein demonstrate similar or superior performance as an absorbent.

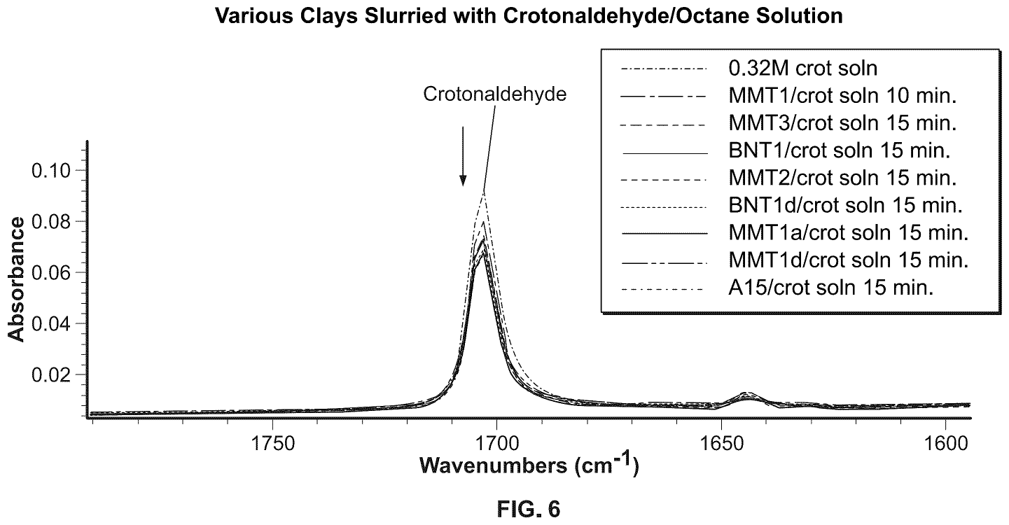

In addition, limited studies were carried out on direct crotonaldehyde adsorption to determine the utility of phyllosilicate clay-based adsorbents for removal of crotonaldehyde as a contaminant. FIG. 6 shows measured absorbance of various clays in 0.32 molar solution of crotonaldehyde in octane after 15 minutes. Overlaid FTIR spectra in FIG. 6. give an indication of the extent of disappearance from solution after 15 minutes in contact with various clays. In all cases, there was no evidence for formation of new peaks in solution. Related quantitative data are shown in Table 5.

TABLE 5

| | | CA Removal |
|---|---|---|
| Ex. | Material | (g/g adsorbent) |
| 16 | MMT1 | 0.047 |
| 17 | MMT1d | 0.090 |
| 18 | MMT1a | 0.090 |
| 19 | MMT2 | 0.071 |
| 20 | MMT3 | 0.065 |
| 21 | BNT1 | 0.068 |
| 22 | BNT1d | 0.072 |
| 23 | A15 | 0.187 |

Comparison of Examples 1-5, 7, and 8 in Table 4 and to Examples 16-22, respectively, in Table 5 indicate that on gram per gram basis, acetaldehyde adsorbs at an order of magnitude greater extent than crotonaldehyde on similar clays.

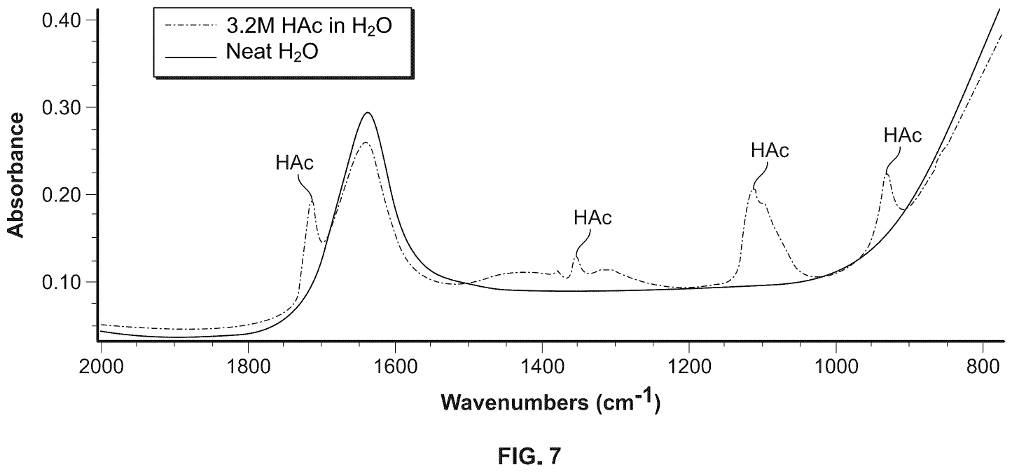

A more limited study was carried out with a stock solution of acetaldehyde in neat H₂O. This water/acetaldehyde stock solution was used as an approximate surrogate for the “decanter light aqueous phase stream,” as described above. Some of the experiments in Table 3 were repeated, but now with a HAc/H₂O stock solution. In this case, a high concentration of acetaldehyde (3.2M, 14.5 wt %) was used to give sufficient FTIR signal. Overlaid FTIR spectra of neat H₂O and of HAc/H₂O are shown in FIG. 7. Adsorption data for these experiments are shown in Table 6. Examples 24-28 show removal capabilities in the in the range of 0.15-0.30 g acetaldehyde/g clay which is lower than in octane solution but still high enough to be considered for acetaldehyde removal from the decanter light aqueous phase stream.

TABLE 6

| | | | | | HAc removed |
|---|---|---|---|---|---|
| Ex. | Clay | Ads | PA | CA | (g/g adsorbent) |
| 24 | MMT1 | Yes | No | No | 0.25 |
| 25 | MMT1a | Yes | No | No | 0.33 |
| 26 | MMT3 | Yes | No | No | 0.15 |
| 27 | MMT3d | Yes | No | No | 0.20 |
| 28 | BNT1 | Yes | No | No | 0.16 |

Finally, a very limited study of crotonaldehyde removal from MeI solution was carried out and the associated data are shown in Table 7. Examples 29-32 again show that adsorption of HAc by the clays are orders of magnitude higher than adsorption of CA.

TABLE 7

| | | CA Removal |
|---|---|---|
| Ex. | Adsorbent | (g/g adsorbent) |
| 29 | MMT1 | 0.004 |
| 30 | MMT1d | 0.024 |
| 31 | BNT1 | 0.012 |
| 32 | A15 | 0.030 |

In summary, useful removal rates of acetaldehyde via adsorption were demonstrated by clays disclosed herein in proxies for either the heavy organic phase stream or the light aqueous phase stream. An appropriate choice of clay will allow useful adsorption rates of acetaldehyde with no unwanted side catalyzed reactions leading to oligomer or polymer formation.

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, methods, and/or steps described in the specification. As one of the ordinary skill in the art will readily appreciate from the disclosure of the present invention, processes, machines, means, methods, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, methods, and/or steps.

What is claimed is:

1. A process for the production of acetic acid, the process comprising:
reacting methanol and carbon monoxide in the presence of a carbonylation catalyst to form acetic acid in an acetic acid production reactor;
flashing a reaction mixture discharged from the acetic acid production reactor into a vapor stream and a liquid stream, the vapor stream comprising acetic acid, water, methanol, methyl acetate, methyl iodide, and acetaldehyde;
separating the vapor stream by distillation in a distillation column into: a product side stream comprising acetic acid and water; a bottoms stream; and an overhead stream comprising methyl iodide, water, methyl acetate, acetic acid, and acetaldehyde;
condensing the overhead stream into: a light aqueous phase stream, comprising methyl iodide, acetaldehyde, water, methyl acetate, and acetic acid; and a heavy organic phase stream, comprising methyl iodide, acetaldehyde, water, methyl acetate, and acetic acid; and
contacting an intermediate process stream with a phyllosilicate clay-based adsorbent at adsorption conditions sufficient to produce a treated intermediate process stream, wherein:
the intermediate process stream comprises at least a portion of the light aqueous phase stream, at least a portion of the heavy organic phase stream, or a combination thereof, and has a first acetaldehyde content;
the treated intermediate process stream has a second acetaldehyde content; and
the second acetaldehyde content is less than the first acetaldehyde content.

2. The process of claim 1, wherein the ratio of the first acetaldehyde content to the second acetaldehyde content is greater than or equal to 2.0.

3. The process of claim 1, further comprising recycling the treated intermediate process stream to the acetic acid production reactor.

4. The process of claim 1, wherein the phyllosilicate clay-based adsorbent comprises a kaolinite, a smectite, a vermiculite, an illite, a chlorite, or a combination thereof.

5. The process of claim 4, wherein the smectite comprises a montmorillonite, beidellite, nantronite, saponite, hectorite, a bentonite, or a combination thereof.

6. The process of claim 1, wherein the phyllosilicate clay-based adsorbent comprises interlayer cations, interlayer metal-oxide pillars, or a combination thereof.

7. The process of claim 1, wherein the phyllosilicate clay-based adsorbent is acidified.

8. The process of claim 1, wherein the phyllosilicate clay-based adsorbent has:
a) a porosity as measured by an average pore volume in the range of from 0.1 to 1.4 cm³/g;
b) a surface area in the range of from 50 m²/g to 800 m²/g;
c) an average removal capability of greater than or equal to 0.3 g acetaldehyde/g of adsorbent; or
d) a combination thereof.

9. The process of claim 1, wherein the phyllosilicate clay-based adsorbent is disposed on a fixed bed.

10. The process of claim 9, wherein the phyllosilicate clay-based adsorbent is loaded in the fixed bed at a level sufficient to provide a loading in a range of from 1 g to 10 g phyllosilicate clay-based adsorbent/g acetaldehyde to be adsorbed.

11. The process of claim 1, wherein the adsorption conditions comprise an adsorption temperature in a range of from room temperature to 250° C.

12. The process of claim 1, wherein the light aqueous phase stream comprises greater than 20 wt % water and the heavy organic phase stream comprises less than 1 wt % water.

13. The process of claim 1, wherein the phyllosilicate clay-based adsorbent is capable of undergoing regeneration.

14. A method for removing acetaldehyde from an acetic acid system, comprising:
providing from the acetic acid system a solution, comprising acetic acid, water, methyl acetate, methyl iodide, and acetaldehyde, wherein the acetaldehyde is present in a first concentration based on the total weight of the solution; and
contacting the solution with a phyllosilicate clay-based adsorbent at adsorption conditions sufficient to produce a treated solution, wherein:
the solution has a first acetaldehyde content;
the treated solution has a second acetaldehyde content; and
the second acetaldehyde content is less than the first acetaldehyde content.

15. The method of claim 14, wherein the ratio of the first acetaldehyde content to the second acetaldehyde content is greater than or equal to 2.0.

16. The method of claim 14, wherein the phyllosilicate clay-based adsorbent comprises a kaolinite, a smectite, a vermiculite, an illite, a chlorite, or a combination thereof.

17. The method of claim 14, wherein the phyllosilicate clay-based adsorbent comprises interlayer cations, interlayer metal-oxide pillars, or a combination thereof.

18. The method of claim 14, wherein the phyllosilicate clay-based adsorbent is acidified.

19. The method of claim 14, wherein the phyllosilicate clay-based adsorbent has:
a) a porosity as measured by an average pore volume in the range of from 0.1 to 1.4 cm³/g;
b) a surface area in the range of from 50 m²/g to 800 m²/g;
c) an average removal capability of greater than or equal to 0.3 g acetaldehyde/g of adsorbent; or
d) a combination thereof.

20. The method of claim 14, wherein the adsorption conditions comprise an adsorption temperature in a range of from room temperature to 250° C.
